(19) United States
(12) Patent Application Publication
(10) Pub. No.: US 2007/0218492 A1
(43) Pub. Date: Sep. 20, 2007

(54) Methods for detecting ovarian cancer and anticipating chemotherapy response

(76) Inventors: Daniel C. Flynn, Morgantown, WV (US); David A. Clump II, Morgantown, WV (US); Jing Jie Yu, Morgantown, WV (US); Eddie Reed, Decatur, GA (US)

Correspondence Address: Craig G. Cochenour, Esq.;Buchanan Ingersoll & Rooney PC, One Oxford Centre, 20th Floor, 301 Grant Street, Pittsburgh, PA, 15219, US

(21) Appl. No.: 11/707,839

(22) Filed: Feb. 16, 2007

Related U.S. Application Data
(60) Provisional application No. 60/773,955, filed on Feb. 16, 2006.

(51) Int. Cl. C12Q 1/68; C12P 19/34

(52) U.S. Cl. 435/6; 435/91.2

(57) ABSTRACT

The disclosure relates to methods of detecting cancer and evaluating risk of cancer. The disclosure also relates to methods of predicting patient response to chemotherapy. The disclosure further relates to the use of a single nucleotide polymorphism in the human AFAP gene for the preparation of a diagnostic compounds for detection of cancer, evaluation of risk of cancer, and prediction of response to chemotherapy.

### CROSS-REFERENCE TO RELATED APPLICATIONS

This application claims priority to pending U.S. Provisional Application No. 60/773,955, filed on Feb. 16, 2006. That application is incorporated by reference as if fully rewritten herein.

### STATEMENT REGARDING FEDERALLY SPONSORED RESEARCH OR DEVELOPMENT

This invention was made, in part, with government support, including one or more of National Institutes of Health grant numbers CA166644 and CA60731. The United States government may have certain rights in this invention.

### BACKGROUND OF THE INVENTION

1. Field of the Invention

The present disclosure relates to novel single nucleotide polymorphisms (SNP) in the human actin filament associated protein (AFAP) gene and the SNPs' association with cancer. The disclosure also relates to use of one or both of these SNPs in predicting and affecting a patient's response to chemotherapy.

2. Background of the Invention.

The recent identification of more than 2 million single nucleotide polymorphisms (SNPs) advances knowledge on the genetic variability between individuals. These SNPs may account for not only differences in personality and physical attributes, but also for differences in disease susceptibility or differences in response to disease treatment. Consequently, these latter attributes make it necessary to identify functional SNPs in order to enhance the personalization of medicine.

One SNP identified by the HapMAP study occurs in the actin filament associated protein of 110 kDa (AFAP-110). AFAP is an SH3/SH2 binding partner for activated Src. Subsequently, AFAP has been characterized as an adaptor protein composed of multiple amino-terminal binding motifs that function upstream of a carboxy-terminal actin binding domain. The functional regions include Src homology domain 2 and 3 binding motifs as well as two pleckstrin homology domains that flank a substrate domain. There is also a leucine zipper that acts in negative regulation of AFAP as it interacts with the PH1 domain stabilizing AFAP as a multimer.

Results conclusively demonstrate that the amino-terminal pleckstrin homology 1 (PH 1) domain interacts with activated PKCct, relaying signals that activate Src family kinases via SH2 and SH3 binding motifs. Serving as an adaptor protein, AFAP is necessary for the assembly of motility structures such as podosomes, lamellipodia, and filopodia in certain model systems.

The cSrc (“Src”) nonreceptor tyrosine kinase is normally repressed and inactive in cells; however, during the G2/M transition, or responsive to growth factor receptor stimulation, Src becomes activated, concomitant with a relaxation of actin filament structures. Src is activated in several human cancer cell lines (Bolen et al., 1987, Proc. Natl. Acad. Sci. USA 84: 2251-2255; Boschek et al., 1981, Cell 24: 175-184; Cartwright et al., 1990, Proc. Natl. Acad. Sci. USA 87: 558-562; Irby et al. 1999, Nat. Genet. 21: 187-190; Rosen et al., 1986, J Biol. Chem. 261: 13754-13759; Tarone et al., 1985, Exp. Cell. Res. 159: 141-157) and one of the hallmarks of transformation by activated forms of Src is the dissolution of stress filaments and a repositioning of actin into rosette-like structures (Reynolds et al., 1989, Mol. Cell. Biol. 9: 3951-3958; Felice et al., 1990, Eur. J Cell Biol. 52: 47-59). Antisense vectors that reduce Src expression in the HT29 human colon cancer cell lines will significantly reduce the transformed properties of these lines and drugs that block Src will impede progression through the G2/M transition. These data demonstrate a role for Src in modulating signals that affect cell growth and motility.

The cSrc proto-oncogene can be activated by dephosphorylation of Tyr⁵²⁷ by cellular phosphatases, or displacement of repressive, intramolecular interactions involving the SH2 and SH3 domains (Brown and Cooper, 1996, Biochim. Biophys. Acta, 1287:121-149). These activation events normally occur in response to cellular signals, e.g., growth factors interacting with their receptors (Brown and Cooper, 1996, supra). These pathways are thought to proceed through Src, with the subsequent phosphorylation of substrates and activation of downstream signaling members, including Ras (He et al., 2000), ppl25^{FAK} (Thomas et al., 1998, Exp. Cell Res., 159:141-157), Crk (Sabe et al., 1992, Mol. Cell Biol., 12: 4706-4713) and ppl30^{Cas} (Xing et al., 2000, Mol. Cell Biol., 20: 7363-7377).

Downstream signaling proteins can modulate the effects of activated Src. For example, Src can be activated by dephosphorylation of Tyr⁵²⁷ by cellular phosphatases, or displacement of repressive, intramolecular interactions involving the SH2 and SH3 domains (Brown and Cooper, 1996, Biochem. Biophys. Acta 1287: 121-149). These activation events usually occur in response to cellular signals, e.g., such as occurs when growth factors interact with their receptors (Brown and Cooper, supra). Activated Src regulates actin filament integrity via signal transduction pathways modulated by downstream effector proteins, including PKCα, PI 3-kinase, Ras (He et al., 2000, Cancer J. 6: 243-248), ppl25FAK (Thomas et al., 1998, J. Biol. Chem. 273: 577-583) Crk (Sabe et al., 1992, supra), Rho and ppl30^{Cas} (Xing et al., 2000, supra). Activated forms of PKCα, PI 3-kinase, and Ras can initiate changes in actin filaments similar to the effects of Src^{527F}. In addition, activation of Src will direct a down-regulation of Rho activity. While dominant negative forms of PKCα, PI 3-kinase, and Ras, will block the effects of Src^{527F} upon actin filaments, dominant-positive forms of Rho will direct the formation of well-formed stress fibers and block the ability of Src^{527F} to alter actin filament integrity.

The actin filament associated protein AFAP-110 is a tyrosine phosphorylated substrate of Src and is an SH2/SH3 binding partner for Src^{527F} (Flynn et al., 1993, Mol. Cell. Biol. 13: 7982-7900). AFAP-110 is an adaptor protein that binds to actin filaments via a carboxy terminal, actin binding domain and colocalizes with stress filaments and the cortical actin matrix along the cell membrane (Quin et al., 1998, Oncogene, 16: 2185-2195; Quin et al., 2000, Exp. Cell. Res., 255:1-2-113). AFAP-110 also is capable of being an SH2/SH3 binding partner for cFyn and cLyn (Flynn et al., 1993, supra; Guappone and Flynn, 1997, Mol. Carinogen. 22: 110-119). In addition to SH2 and SH3 binding motifs, AFAP comprises two pleckstrin homology domains (PH1 and PH2), a carboxy terminal leucine zipper, which facilitates self association of AFAP-110 (Quin et al., 1998, supra) and an actin binding domain (Flynn et al., supra, Qian et al, 2000, supra). AFAP-110 also contains a target region for serine/threonine phosphorylation as well as other hypothetical protein-binding sites (Baisden et al., 2001a, Oncogene, 20:6435-6447). AFAP-110 is hyperphosphorylated on ser/thr residues as well as tyrosine residues in Src transformed cells and contains numerous consensus sequences for phosphorylation by PKC (Kanner et al., 1991, EMBO J., 10:1689-1698; Flynn et al., 1993, supra). AFAP-110 appears to function as an adapter molecule linking a variety of signaling proteins to the actin cytoskeleton.

The carboxy terminal leucine zipper motif appears to play a regulatory role for AFAP-110. In Src^{527F} transformed cells, AFAP-110 undergoes a conformational change that affects its ability to self-associate via the leucine zipper motif (Qian et al., 1998 supra). This conformational change was detected using two separate assays that demonstrate changes in self-association, affinity chromatography (affinity absorptions) and gel filtration analysis. Affinity absorption of AFAP-110 using the GST-cterm fusion proteins that encoded the leucine zipper motif, will bind cellular AFAP-110. Deletion of the leucine zipper motif from GST-cterm prevents affinity absorption. In Src^{527F} transformed cell, GST-cterm can no longer bind cellular AFAP-110, indicating that Src^{527F} directed cellular signals which affected a change in AFAP-110 conformation, precluding access to an intramolecular binding site for the leucine zipper motif. Gel filtration analysis confirmed that cellular AFAP-110 does self-associate, existing predominantly as a multimer (trimers, tetramers and possibly larger complexes), with a minor population predicted to be monomers. In Src^{527F}-transformed cells, AFAP-110 fractionated as a single population, predicted to represent dimers. Thus, Src^{527F} directed a cellular signal that alters AFAP-110 conformation. The cellular signal that affected this change in conformation of AFAP-110 appeared to occur independently of tyrosine phosphorylation. Co-expression of cSrc had no effect on the ability of GST-cterm to affinity absorb AFAP-110, even though cSrc was able to direct tyrosine phosphorylation of AFAP-110 (Qian et al., 1998 supra).

The functional significance of the leucine zipper motif was revealed by deletional mutagenesis and expression of AFAP-110^{Δlzip} and expression of AFAP-110^{Δlzip} in fibroblast cell lines. Here, AFAP-110^{Δlzip} induced a phenotype similar to Src^{527F}, resulting in a repositioning of actin filaments into rosette-like structures and the formation of motility structures (Qian et al., 1998 supra; Qian et al., 2000 supra). Subsequent data demonstrated that the mechanism by which AFAP-110^{Δlzip} was able to alter actin filament integrity was via its ability to activate cellular tyrosine kinases. However, engineering of a point mutation into AFAP-110^{Δlzip} that abrogated SH3 binding to Src (Pro⁷¹→Ala⁷¹; Guappone and Flynn, 1997 supra), prevented AFAP-110^{Δlzip/71A} from altering actin filament integrity. Previously, it had been shown that Src family kinases can be activated by engagement with SH3 binding partners. Activation is achieved by interfering with and relieving the repressive intramolecular interactions involving the SH3 domain of cSrc. The HIV Nef protein and Herpesvirus Tip protein have been shown to activate Src family members in an SH3-dependent fashion (Collette et al., 2000; Hartley et al., 1999; Moarefi et al., 1997). Both of these proteins transform cells, and the Tip protein is a requirement for the induction of tumors by the Herpesvirus (Duboise et al., 1998). The ability of AFAP-110^{Δlzip} to alter actin filament integrity was due to the subsequent activation of Rho, downstream of activated cSrc. Dominant positive RhoA^{V14} was able to impede the ability of AFAP-110^{Δlzip} from altering actin filament integrity, while being unable to effect induction of cSrc family activation or cellular tyrosine phosphorylation. These data indicated that AFAP-110^{Δlzip} was directing changes in actin filament integrity via SH3 mediated activation of cSrc with subsequent stimulation to the Rho family, which are well known effectors of actin filament integrity (Ridley 1992, Prog. Mol. Subcell. Biol., 22:1-22). Thus, it was hypothesized that AFAP-110 is a cSrc activating protein and the integrity of the leucine zipper motif was important for regulating this function. Changes in conformation could present AFAP-110 as an efficient SH3 binding partner for cSrc, enabling SH3-mediated activation of cSrc and subsequent changes in actin filament integrity.

### BRIEF SUMMARY OF THE INVENTION

Embodiments of the invention relate to methods of detecting cancer and evaluating risk of cancer. Embodiments also relate to methods of predicting and/or enhancing patient response to chemotherapy. Embodiments further relate to the use of one or more single nucleotide polymorphisms in the human AFAP gene for the preparation of a diagnostic compounds for detection of cancer, evaluation of risk of cancer, and prediction of response to chemotherapy.

Despite published results indicating the function of the PH 1 domain, little is known about the PH2 domain of AFAP. The PH2 domain is located between position 347 and 450 of this 729 amino acid protein. In addition to the HapMAP study (rs28406288), a database analysis of the various transcripts of human AFAP reveals that an alternative sequence for AFAP exists (BC032777). This alternative sequence demonstrates a nucleotide change of G1208C resulting in an amino acid change of serine (S) to cysteine (C) at position 403. Therefore, this change identifies the possibility of a single nucleotide polymorphism (SNP) in AFAP. A second SNP present in the human AFAP gene with a high correlation to G1208C is G297A, which is a silent mutation that does not change the proline at position 99. Unless otherwise indicated, references herein to “the SNP” shall refer to the G1208C SNP.

AFAP is intricately linked with Src family kinase activity, which is a characteristic of the transformed cell phenotype. It is well known that Src family kinase activity is increased in large percentage of carcinomas, including the predominant kinase involved in ovarian cancer. It is therefore appealing to determine the interaction between the AFAP SNP and this kinase. This work details the relationship between AFAP 403S/C and Src in the context of ovarian cancer.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1A shows the coding sequence of human AFAP-110 from 5′ to 3′ (SEQ ID NO: 1). Start and stop codons, respectively, are underlined. FIG. 1B shows the intron-exon structure of human AFAP-110. FIGS. 1A and 1B are directed to the AFAP-110 gene without the SNPs described herein.

FIG. 2 shows the amino acid sequence of human AFAP-110 (SEQ ID NO: 2). The positions of intron-exon boundaries are indicated. The amino acid sequence of FIG. 2 is shown without the mutation resulting from the SNP described herein.

FIG. 3 shows the coding sequence of AFAP-110 from 5′-3′, including the two SNPs described herein (SEQ ID NO: 3). Start and stop codons, respectively, are underlined.

FIG. 4 shows the amino acid sequence of AFAP-110 including the Ser403→Cys403 mutation resulting from the SNP described herein (SEQ ID NO: 4).

FIG. 5 is a schematic of the AFAP-110 amino acid structure. AFAP has been characterized as an adaptor protein composed of multiple amino-terminal binding motifs that function upstream of a carboxy-terminal actin binding domain.

FIG. 6 is a model of AFAP signaling. Serving as an adaptor protein, AFAP is necessary for the assembly of motility structures such as podosomes, lamellipodia, and filopodia in certain model systems.

FIG. 7 is a representation of PH2 wild-type versus the disulfide bond shift in the PH2 with a cysteine instead of the conventional serine. The cysteine resulting from the SNP exists in a loop region between the 5th and 6th β-sheet of the PH2 domain of AFAP. Here, a base pair change of G to C at position 1208 results in an amino acid change of serine to cysteine. Functionally, both serine and cysteine are classified as polar, uncharged amino acids. However, the presence of an additional cysteine in the primary structure could potentially alter protein folding via the formation of disulfide bonds. Molecular modeling suggests that wild-type AFAP has a single disulfide bond in the PH2 domain existing between the first and second cysteine, which is the first and second β-sheet. However, the SNP in AFAP is predicted to alter disulfide bond formation to occur between the first and third cysteine, which is between the first and fifth β-sheet on opposing faces of the PH domain. Consequently, the tertiary structure of AFAP could be altered. This could potentially result in altered protein or lipid binding partners.

FIG. 8 shows a serine to cysteine change made by converting the codon TCA to TGT in wild-type AFAP at position 403. AFAP constructs were then transiently transfected into SYFcSrc cells. Cellular effects were determined by analyzing actin stress filament integrity and podosome formation distinguished by cortactin colocalization. FIG. 8 also shows a Src family kinase activity determined by phosphorylation at tyrosine 416. Results demonstrate that the AFAP 403S/C SNP colocalizes with cortactin and actin in podosomes without prior activation C and similar to that of dominant-positive AFAP ΔLzip. In addition, AFAP 403S/C activation of Src without prior activation.

FIG. 9 shows PMA direction of AFAP-110 to colocalize with and activate cSrc.

FIG. 10 is a PCR analysis of AFAP sequence in ovarian cancer cell lines, as well as ovarian cancer tissue sections reveal that this SNP exists in approximately 25% of the samples.

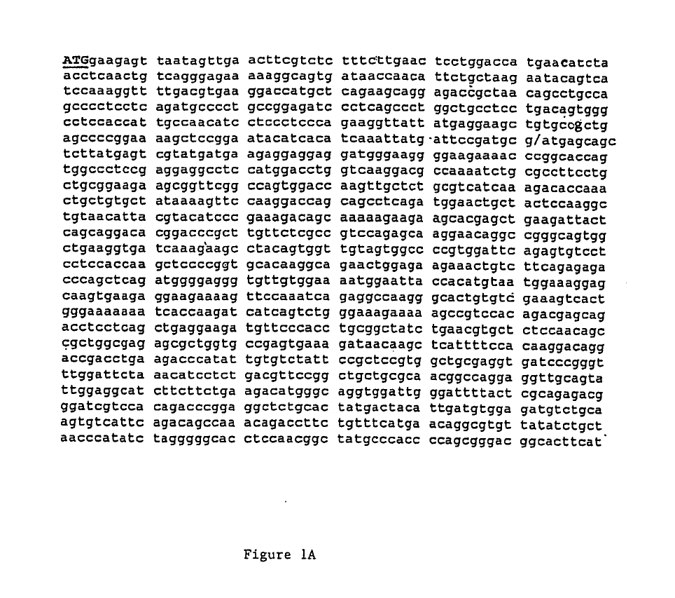

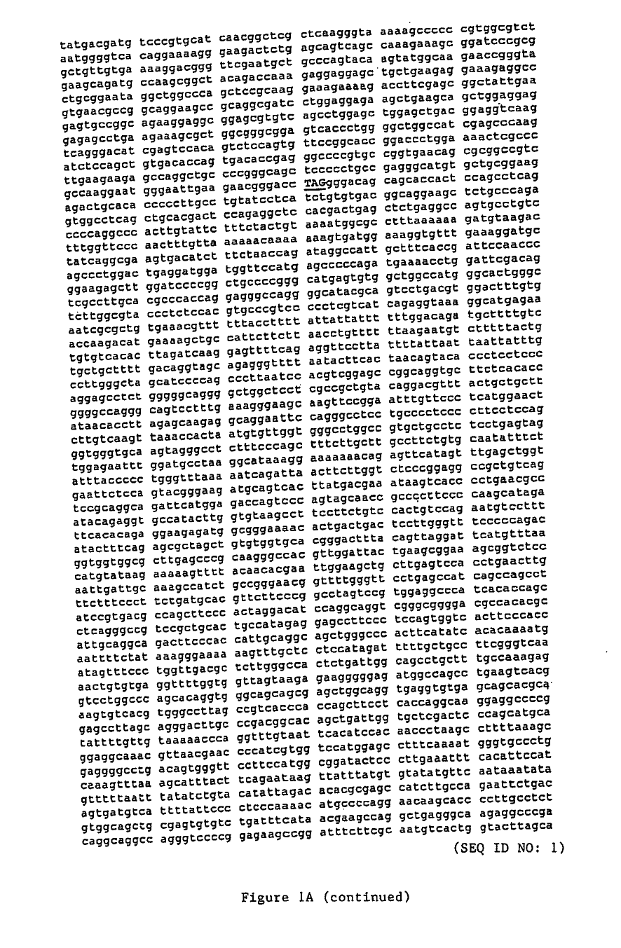

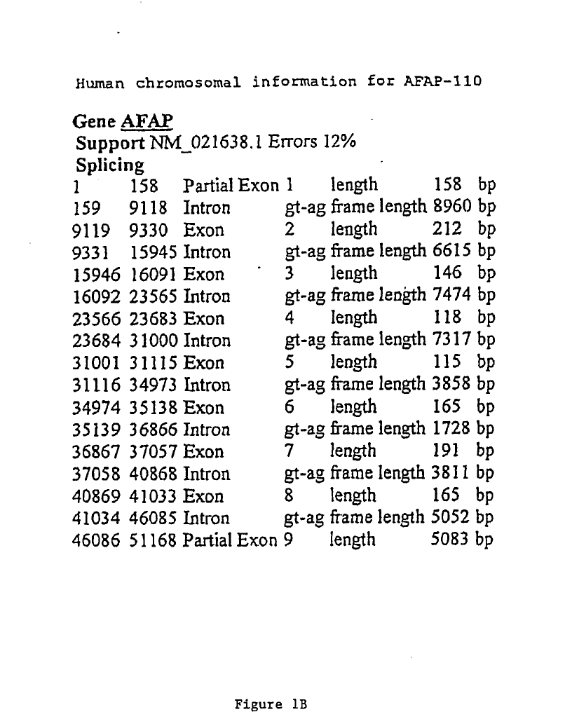

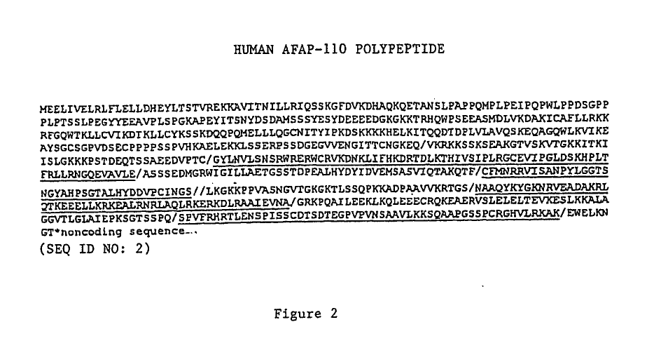

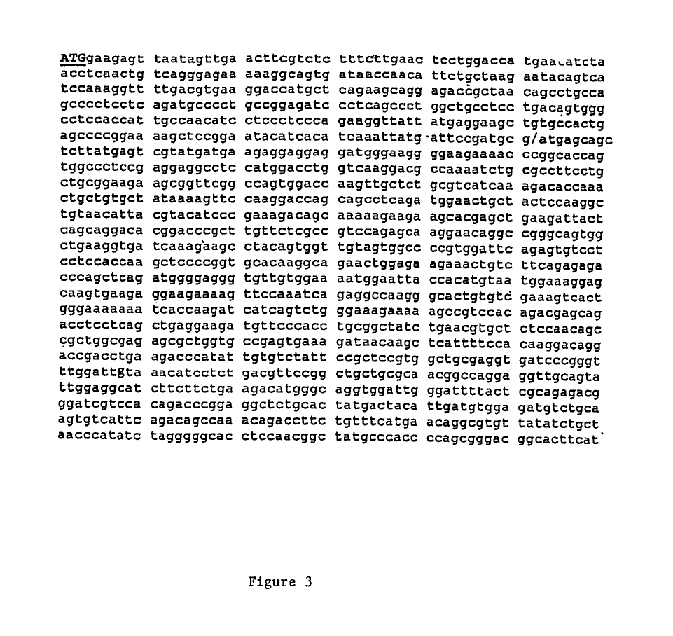

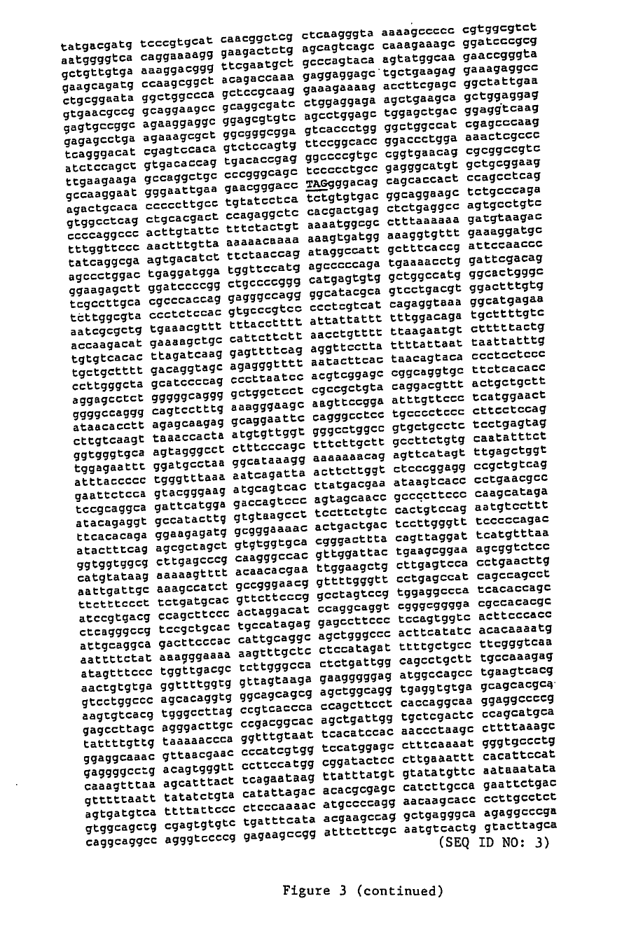

### DETAILED DESCRIPTION OF THE INVENTION

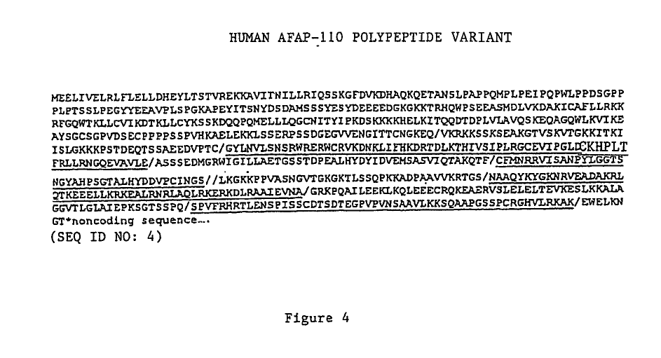

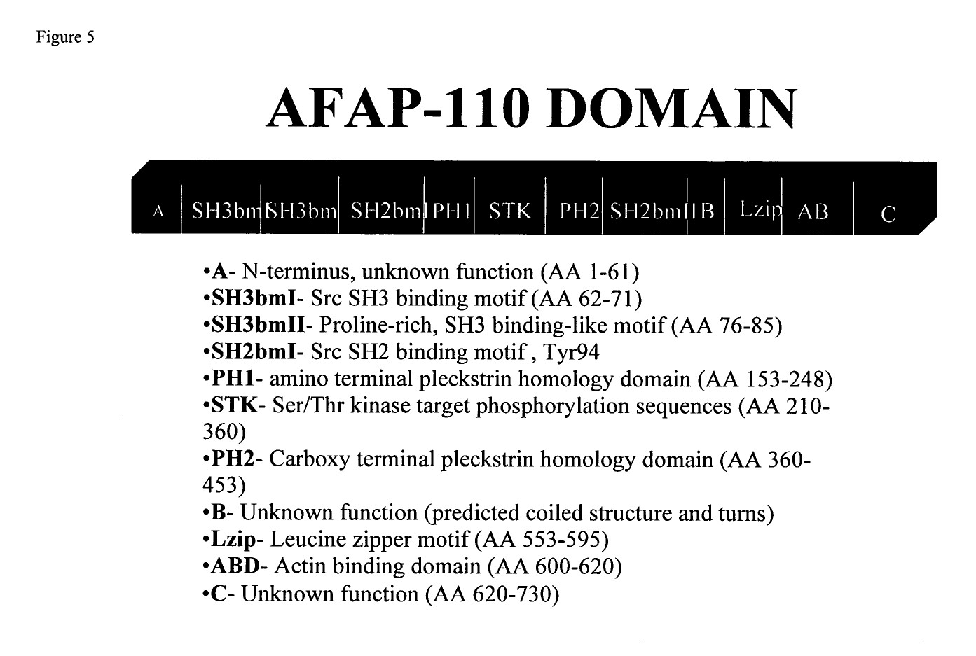

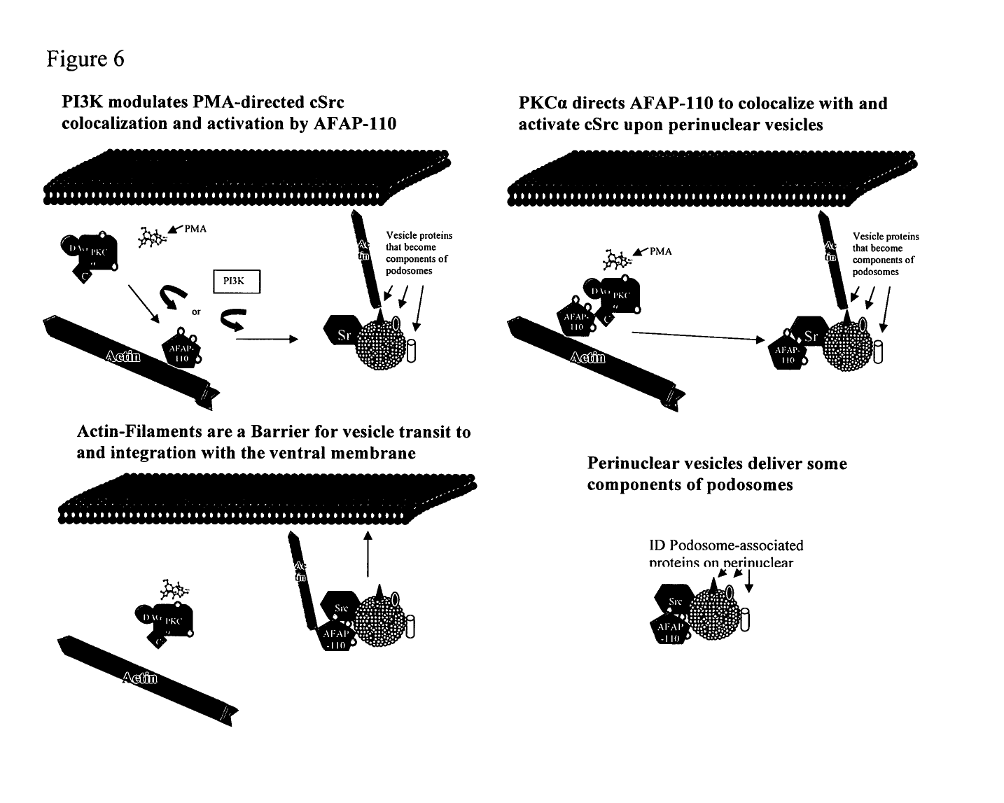

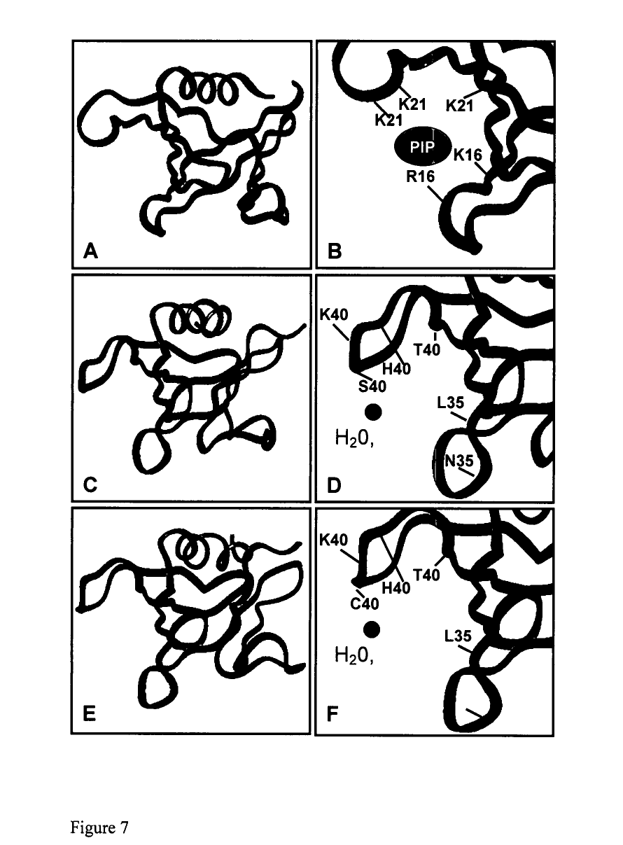

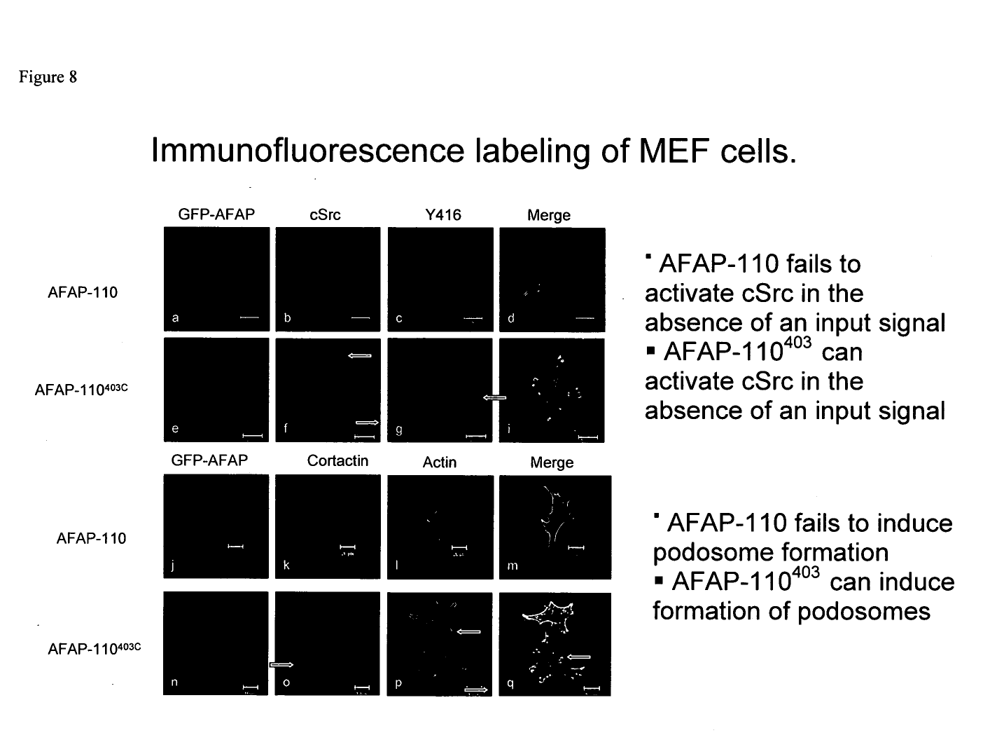

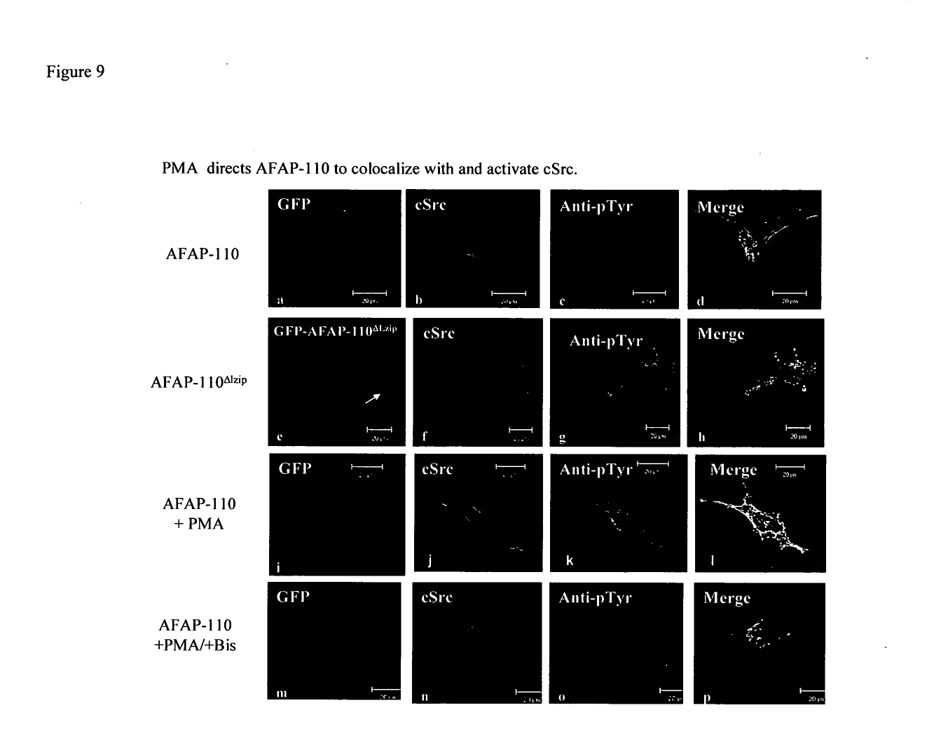

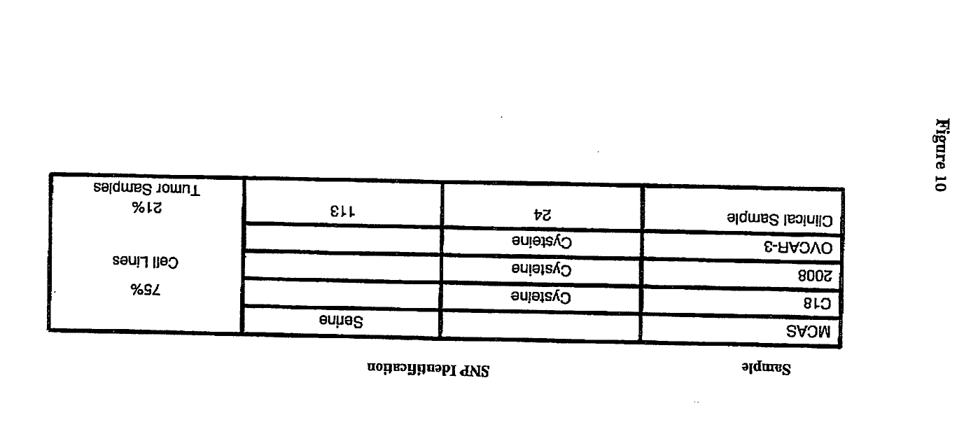

We have discovered a single nucleotide polymorphism (SNP) at codon 403 in the coding sequence of AFAP. The SNP involves a single nucleotide substitution, resulting in a C→G transition (TCT→TGT), thereby changing the predicted amino acid at the 403 position from serine (Ser403) to cysteine (Cys403), resulting in the polymorphic variant of AFAP-110 shown in FIG. 4. We have also discovered a SNP at codon 99 in the coding sequence of AFAP (CCG→CCA) that is a silent mutation. There is a high correlation between the occurrence of the two SNPs. The detection of the Ser/Cys substitution polymorphic variant in ovarian cancer cell lines in three of four cases (75%) suggests the occurrence of a novel SNP in the AFAP gene. As shown in Table 1, occurrence of this polymorphic variant was also observed in ovarian tumors in 27 of 140 cases (19%).

TABLE 1
DNA Sequencing Results of AFAP-110 in Ovarian Tumor Specimens
Mutation (1208 bp)

| | TCT to TGT |
|---|---|
| Sample # | Serine → Cysteine |
| (Tumorous tissue) 91 | 18 |
| (Normal tissue, adjacent) 49 | 9 |
| Total 140 | 27 |

The cSrc protein is expressed at high levels in ovarian cancer, as well as breast, gastrointestinal, brain, and other soft tissue cancers. The cSrc protein is activated as an enzyme. Although wild type AFAP did not activate cSrc without an input signal, the polymorphic variant activated cSrc constitutively. Moreover, the polymorphic variant was overexpressed in many of the ovarian tumor samples studied. Overexpression of the polymorphic variant directed activation of cSrc, while overexpression of the wild type did not. Activation of cSrc renders tumors invasive and resistant to chemotherapy.

One embodiment of the invention provides a method for screening a subject having cancer or at risk of having cancer by analysis of an SNP in the AFAP gene. The cancer may be, for example, but is not limited to, ovarian cancer, breast cancer, or gastrointestinal cancer cancer. Preferably the cancer is ovarian cancer. The method comprises the steps of:

(a) providing a sample of a target nucleic acid from a subject; and

(b) amplifying the target nucleic acid by polymerase chain reaction (PCR) using oligonucleotide primers, wherein the target nucleic acid is amplified only if the SNP in the AFAP gene leading to Ser403→Cys403 is present, and wherein presence of the SNP is indicative of cancer or risk of cancer. In a further embodiment the target nucleic acid is amplified only if the SNP G297A is present in the AFAP gene. In a still further embodiment, both the G297A SNP and the C1208G SNP must be present for the target nucleic acid to be amplified.

In one embodiment, the sample of target nucleic acid is mRNA. In a further embodiment, the sample is cDNA. In a further embodiment the sample is isolated from a tumor tissue. In a yet still further embodiment, the oligonucleotide primers are AFAP-F3: 5′-gaaagaaaaagccgtccacagacgag-3′ (SEQ ID NO: 5) and AFAP-R3: 5′-ttgagcgagccgttgatgcacgg-3′ (SEQ ID NO: 6).

In a another embodiment, the method further comprises the step of isolating a sample of a target nucleic acid from a tissue of interest. A tissue of interest may be, for example, ovarian tissue, breast tissue, buccal tissue, tumor tissue, or normal (i.e. non-tumorous) tissue.

Where the SNP is present, a subject may undergo further screening. For example, if the SNP is present in buccal cells of a woman at risk for ovarian cancer, that woman may be checked annually by another method to determine if the woman has early disease. That method may be, for example, pelvic exam or transvaginal ultrasound.

In yet another embodiment, the method further comprises the step of sequencing the PCR products of step (b) to confirm the existence of the SNP resulting in Ser403→Cys403. In a further embodiment, the PCR products are amplified to confirm the existence of the G297A SNP. In a still further embodiment, the existence of both SNPs is confirmed. DNA sequencing may be performed, for example, by chain termination, pyrosequencing, 454 Sequencing, nanopore sequencing, hybridization sequencing, and polony sequencing. Those skilled in the art will recognize a number of other conventional sequencing methods that may be used.

The presence of polymorphic AFAP in a newly diagnosed tumor indicates that cSrc may be activated. Because activation of cSrc is known to result in resistance to chemotherapy, diagnosis of activated cSrc allows a patient to be placed on a treatment regimen including a therapeutic amount of a drug that targets cSrc. One drug that may be used, for example, is the tyrosine kinase inhibitor dasatinib (Bristol-Myers Squibb).

One embodiment of the invention provides a method for screening a subject for prediction of response to chemotherapy by analysis of an SNP in the AFAP gene. The method comprises the steps of:

(a) providing a sample of a target nucleic acid from a subject; and

(b) amplifying the target nucleic acid by polymerase chain reaction (PCR) using oligonucleotide primers, wherein the target nucleic acid is amplified only if the SNP in the AFAP gene leading to Ser403→Cys403 is present, only if the G297A SNP in the AFAP gene is present, or only if both SNPs are present and wherein presence of the selected SNPs is indicative of activation of cSrc and lack of response to chemotherapy.

In one embodiment, the sample of target nucleic acid is mRNA. In a further embodiment, the sample is cDNA. In a further embodiment the sample is isolated from a tumor tissue. In a yet still further embodiment, the oligonucleotide primers are AFAP-F3: 5′-gaaagaaaaagccgtccacagacgag-3′ (SEQ ID NO: 5) and AFAP-R3: 5′-ttgagcgagccgttgatgcacgg-3′ (SEQ ID NO: 6).

In a another embodiment, the method further comprises the step of isolating a sample of a target nucleic acid from a tissue of interest. A tissue of interest may be, for example, ovarian tissue, breast tissue, buccal tissue, tumor tissue, or normal (i.e. non-tumorous) tissue.

In yet another embodiment, the method further comprises the step of sequencing the PCR products of step (b) to confirm the existence of the SNP resulting in Ser403→Cys403, the existence of the G297A SNP or both. DNA sequencing may be performed, for example, by chain termination, pyrosequencing, 454 Sequencing, nanopore sequencing, hybridization sequencing, and polony sequencing. Those skilled in the art will recognize a number of other conventional sequencing methods that may be used.

In a further embodiment, the invention provides a method of increasing patient response to chemotherapy, comprising the steps of:

(a) providing a sample of a target nucleic acid from a subject;

(b) amplifying the target nucleic acid by polymerase chain reaction (PCR) using oligonucleotide primers, wherein the target nucleic acid is amplified only if the SNP in the AFAP gene leading to Ser403→Cys403 is present, only if the G297A SNP in the AFAP gene is present, or only if both SNPs are present, and wherein presence of the SNP is indicative of activation of cSrc; and

(c) administering to said subject a therapeutic amount of a compound that deactivates cSrc, wherein deactivation of cSrc increases patient response to chemotherapy.

In one embodiment, the sample of target nucleic acid is mRNA. In a further embodiment, the sample is cDNA. In a further embodiment the sample is isolated from a tumor tissue. In a yet still further embodiment, the oligonucleotide primers are AFAP-F3: 5′-gaaagaaaaagccgtccacagacgag-3′ (SEQ ID NO: 5) and AFAP-R3: 5′-ttgagcgagccgttgatgcacgg-3′ (SEQ ID NO: 6). In a another embodiment, the method further comprises the step of isolating a sample of a target nucleic acid from a tissue of interest. A tissue of interest may be, for example, ovarian tissue, breast tissue, buccal tissue, tumor tissue, or normal (i.e. non-tumorous) tissue.

In yet another embodiment, the method further comprises the step of sequencing the PCR products of step (b) to confirm the existence of the SNP resulting in Ser403→Cys403, the existence of the G297A SNP, or both. DNA sequencing may be performed, for example, by chain termination, pyrosequencing, 454 Sequencing, nanopore sequencing, hybridization sequencing, and polony sequencing. Those skilled in the art will recognize a number of other conventional sequencing methods that may be used. Those skilled in the art will recognize that the detection of the SNP may be performed at least two ways. In one embodiment, a primer is used that will only amplify DNA containing the SNP; if the DNA is amplified, the SNP is present. The amplified DNA may optionally be sequenced to confirm the presence of the SNP. In another embodiment, a primer is used that will amplify AFAP DNA regardless of the presence of the SNP. The amplified DNA is then sequenced to determine if the SNP is present.

The AFAP polymorphic variant can also be detected. A method for detecting the AFAP polymorphic variant comprises the steps of:

(a) providing a sample of a protein;

(b) determining the amino acid sequence of said protein;

(c) comparing the amino acid sequence of said protein with the amino acid sequence of the AFAP polymorphic variant including the 403S/C mutation, wherein identical sequences indicate the presence of the AFAP polymorphic variant.

Where the polymorphic variant is detected, treatment of a subject may begin in the same manner as when the nucleic acid sequence leading to the polymorphic variant is detected, as discussed above.

Embodiments herein may start with a PCR analysis of ovarian cancer cell lines and ovarian cancer tissue. RNA was then isolated and converted to cDNA for PCR analysis. PredictProtein software (Columbia University) was used to predict structure of PH2 domain compared to that of the PH2 S/C SNP. The primary sequence and tertiary structure was predicted using PROF predictions and Disulfind programs. The AFAP-403S/C-GFP was created by base pair mutagenesis of avian AFAP-110-GFP construct. A serine to cysteine change was made by converting the codon TCA to TGT. This effectively created a restriction fragment length polymorphism (RFLP) by removing a Hinfl site at base pair 1222. Next, the transient transfections of SYF-cSrc (ATCC) were performed with AFAP-GFP constructs using Lipofectamine and Plus reagent (Invitrogen) as per manufacturer specifications. Immunoflurescent staining using the following primary antibodies: polyclonal cortactin antibody (AbCAM 1:1000), TRITC-phalloidin (Sigma 1:500), polyclonal Y416 antibody (Cell Signaling 1:500), Src monoclonal antibody EC 10 (t:1000), and the secondary antibodies anti-Rabbit Alexa 647 and anti-Mouse Alexa 538 (Molecular Probes 1:200) were performed. Images retrieved using Zeiss LSMS10 confocal microscope.

After the experimentation, the following is now known or can be predicted from this invention. PCR analysis of AFAP sequence in ovarian cancer cell lines, as well as ovarian cancer tissue sections reveal that this SNP exists in approximately 25% of the samples. The mutation resulting from the described SNP exists in a loop region between the 5th and 6th β-sheet of the PH2 domain of AFAP. Here, a base pair change of G to C at position 1208 results in an amino acid change of serine to cysteine. Functionally, both serine and cysteine are classified as polar, uncharged amino acids. However, the presence of an additional cysteine in the primary structure could potentially alter protein folding via the formation of disulfide bonds. Molecular modeling suggests that wild-type AFAP has a single disulfide bond in the PH2 domain existing between the first and second cysteine, which is the first and second β-sheet. However, the SNP in AFAP is predicted to alter disulfide bond formation to occur between the first and third cysteine, which is between the first and fifth β-sheet on opposing faces of the PH domain. Consequently, the tertiary structure of AFAP could be altered. This could potentially result in altered protein or lipid binding partners.

To test the effects of this SNP, a serine to cysteine change was made by converting the codon TCA to TGT in wild-type AFAP at position 403. AFAP constructs were then transiently transfected into SYFcSrc cells. Cellular effects were determined by analyzing actin stress filament integrity and podosome formation distinguished by cortactin colocalization. In addition, Src family kinase activity was determined by phosphorylation at tyrosine 416. Results demonstrate that the AFAP 403S/C SNP colocalizes with cortactin and actin in podosomes without prior activation with PKC and similar to that of dominant-positive AFAP ALzip. In addition, AFAP 403S/C induces activation of Src without prior activation. These results lead to the following conclusions.

First, the AFAP SNP exists in 20% of ovarian cancer tissue samples and, therefore, could be found in any solid tumor including breast, lung, colon, and brain cancers. Next, the SNP is characterized by a serine to cysteine change that potentially alters the tertiary structure of the PH2 domain at position 403. Finally, this SNP may prime the cell for invasion by promoting podosome formation via activation of cSrc.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

### EXAMPLES

Methods of embodiments of the invention are illustrated by the following examples, which should not be construed as limitations on the scope of the invention.

cDNA Preparation and PCR Amplification

One hundred forty normal and tumor specimens from patients with advanced ovarian cancer in stage 3 or stage 4 were obtained from CHTN (Cooperative Human Tissue Network), Pediatric Division, Children's Hospital, Columbus, Ohio. Samples were collected prior to drug treatment and snap-frozen at −80° C. until RNA/DNA extraction. All specimens were diagnosed and classified by pathologists. From each of the 140 specimens, total cellular RNA was isolated and purified by the method of hot phenol/chloroform extraction. Purified RNAs were precipitated and dissolved in DEPC water. Through reverse transcription, using the SuperScript Preamplification System, cDNAs were generated with oligo-dT primers from 5 μg of total RNA per sample (Reverse Transcription System, Promega, Madison, Wis.).

AFAP Gene Amplification by Polymerase Chain Reaction (PCR)

With selected exon-primers for the AFAP, cDNA was amplified by polymerase chain reaction (PCR) with AmpliTaq DNA polymerase, FS under optimal condition on GeneAmp 9600 (Applied Biosystems). The PCR conditions were 95° C. for 3 minutes flowed by 40 cycles of 95 ° C. for 30 seconds, 58 ° C. for 30 seconds and 72 ° C. for 45 seconds; and then 1 cycle of 72 ° C. for 10 minutes. The primer sequences are AFAP-F3: 5′-gaaagaaaaagccgtccacagacgag-3′ (SEQ ID NO: 5) and AFAP-R3: 5′-ttgagcgagccgttgatgcacgg-3′ (SEQ ID NO: 6).

DNA Sequencing

PCR amplicons were separated on agarose gel and purified using QIAQuick Gel Extraction Kit (GIAGEN). The purified fragments were sequenced to identify AFAP mutations using the CEQ 8000 Genetic Analysis System with GenomeLab DTCS-Quick Start Kit (Beckman Coulter) and then using ABI Prism DNA Sequencer with BigDye Terminator Cycle Sequencing Kit (Applied Biosystems) to confirm identified mutations. All sequence variants were confirmed in duplicate independent PCR amplifications and sequencing reactions to insure that the mutations was not a result of PCR artifact.

All claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, and the claims referred to above including but not limited to any original claims.

As used herein and in the appended claims, the singular forms “a,” “an,” and “the” include plural reference unless the context clearly dictates otherwise.

The terms and expressions employed herein have been used as terms of description and not of limitation, and there is no intention in the use of such terms and expressions, or any portions thereof, to exclude any equivalents now know or later developed, whether or not such equivalents are set forth or shown or described herein or whether or not such equivalents are viewed as predictable, but it is recognized that various modifications are within the scope of the invention claimed, whether or not those claims issued with or without alteration or amendment for any reason. Thus, it shall be understood that, although the present invention has been specifically disclosed by preferred embodiments and optional features, modifications and variations of the inventions embodied therein or herein disclosed can be resorted to by those skilled in the art, and such modifications and variations are considered to be within the scope of the inventions disclosed and claimed herein.

Specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. Where examples are given, the description shall be construed to include but not to be limited to only those examples. It will be readily apparent to one skilled in the art that varying substitutions and modifications may be made to the invention disclosed herein without departing from the scope and spirit of the invention, and from the description of the inventions, including those illustratively set forth herein, it is manifest that various modifications and equivalents can be used to implement the concepts of the present invention without departing from its scope. A person of ordinary skill in the art will recognize that changes can be made in form and detail without departing from the spirit and the scope of the invention. The described embodiments are to be considered in all respects as illustrative and not restrictive. Thus, for example, additional embodiments are within the scope of the invention and within the following claims.

We claim:

1. A method of diagnosing an increased risk to develop cancer in a subject comprising:
(a) determining the presence or absence of at least one single nucleotide polymorphism in the AFAP gene in a biological sample; and
(b) diagnosing an increased risk to develop cancer based on the presence of a single nucleotide polymorphism in the AFAP gene.

2. The method of claim 1, wherein said single-nucleotide polymorphism is at least one member selected from the group consisting of a codon 403 [TCT→TGT ] transition in the human AFAP gene (SEQ ID NO: 1) and a codon 99 [CCG→CCA] transition in the human AFAP gene (SEQ ID NO: 1).

3. The method of claim 1, wherein said single-nucleotide polymorphism is selected from the group consisting of a codon 403 [TCT→TGT] transition in the human AFAP gene (SEQ ID NO: 1).

4. A method of diagnosing cancer in a subject comprising:
(a) determining the presence or absence of at least one single nucleotide polymorphism in the AFAP gene in a biological sample; and
(b) diagnosing a cancer based on the presence of a single nucleotide polymorphism in the AFAP gene.

5. The method of claim 4, wherein said single-nucleotide polymorphism is at least one member selected from the group consisting of a codon 403 [TCT→TGT] transition in the human AFAP gene (SEQ ID NO: 1) and a codon 99 [CCG→CCA] transition in the human AFAP gene (SEQ ID NO: 1).

6. The method of claim 5, wherein said single-nucleotide polymorphism is a codon 403 [TCT→TGT] transition in the human AFAP gene.

7. A method of diagnosing resistance to chemotherapy in a subject comprising:
(a) determining the presence or absence of a single nucleotide polymorphism in the AFAP gene in a biological sample; and
(b) diagnosing resistance to chemotherapy based on the presence of a single nucleotide polymorphism in the AFAP gene.

8. The method of claim 7, wherein said single-nucleotide polymorphism is at least one member selected from the group consisting of a codon 403 [TCT→TGT] transition in the human AFAP gene (SEQ ID NO: 1) and a codon 99 [CCG→CCA] transition in the human AFAP gene (SEQ ID NO: 1).

9. The method of claim 8, wherein said single-nucleotide polymorphism is a codon 403 [TCT→TGT] transition in the human AFAP gene.

10. A method for detecting a single nucleotide polymorphism (SNP) in human subjects having or at risk of having cancer, said (SNP) being indicative of risk of cancer, presence of cancer, and/or resistance to chemotherapy, the method comprising:
(a) providing a tissue sample from a subject;
(b) isolating a target mRNA from said tissue sample;
(c) preparing a cDNA copy of said target mRNA;
(d) attempting to amplify said cDNA with the polymerase chain reaction, wherein said amplification occurs only if said cDNA includes a target SNP.

11. The method of claim 10, wherein said cancer is selected from the group consisting of breast cancer, ovarian cancer, and gastrointestinal cancer.

12. The method of claim 10, further comprising the step of determining the sequence of said cDNA.

13. The method of claim 10, wherein said target SNP is a single nucleotide polymorphism in a human AFAP gene (SEQ ID NO: 1).

14. The method of claim 13, wherein said single-nucleotide polymorphism is at least one member selected from the group consisting of a codon 403 [TCT→TGT] transition in the human AFAP gene (SEQ ID NO: 1) and a codon 99 [CCG→CCA] transition in the human AFAP gene (SEQ ID NO: 1).

15. The method of claim 14, wherein said target SNP is a codon 403 [TCT→TGT] transition.

16. The method of claim 8, wherein said amplification is attempted using by PCR, using the primers AFAP-F3: 5′-gaaagaaaaagccgtccacagacgag-3′ (SEQ ID NO: 5) and AFAP-R3: 5′-ttgagcgagccgttgatgcacgg-3′ (SEQ ID NO: 6).

17. A method of diagnosing cancer, an increased risk to develop cancer, and/or resistance to chemotherapy in a subject comprising:
(a) determining the presence or absence of a human AFAP gene variant in a biological sample, wherein said variant comprises at least one single nucleotide polymorphism; and
(b) diagnosing cancer, an increased risk to develop cancer, and/or resistance to chemotherapy based on the presence of a human AFAP variant.

18. The method of claim 17, wherein said human AFAP gene variant has an amino acid sequence of SEQ ID NO: 4.


SEQUENCE LISTING (6 sequences)

SEQ ID NO: 1 - DNA, 5040 bp, Homo sapiens
atggaagagttaatagttgaacttcgtctctttcttgaactcctggaccatgaatatctaacctcaactgtcagggagaaaaaggcagtgataaccaacattctgctaagaatacagtcatccaaaggttttgacgtgaaggaccatgctcagaagcaggagaccgctaacagcctgccagcccctcctcagatgcccctgccggagatccctcagccctggctgcctcctgacagtgggcctccaccattgccaacatcctccctcccagaaggttattatgaggaagctgtgccgctgagccccggaaaagctccggaatacatcacatcaaattatgattccgatgcgatgagcagctcttatgagtcgtatgatgaagaggaggaggatgggaaggggaagaaaacccggcaccagtggccctccgaggaggcctccatggacctggtcaaggacgccaaaatctgcgccttcctgctgcggaagaagcggttcggccagtggaccaagttgctctgcgtcatcaaagacaccaaactgctgtgctataaaagttccaaggaccagcagcctcagatggaactgctactccaaggctgtaacattacgtacatcccgaaagacagcaaaaagaagaagcacgagctgaagattactcagcaggacacggacccgcttgttctcgccgtccagagcaaggaacaggccgggcagtggctgaaggtgatcaaagaagcctacagtggttgtagtggccccgtggattcagagtgtcctcctccaccaagctccccggtgcacaaggcagaactggagaagaaactgtcttcagagagacccagctcagatggggagggtgttgtggaaaatggaattaccacatgtaatggaaaggagcaagtgaagaggaagaaaagttccaaatcagaggccaagggcactgtgtcgaaagtcactgggaaaaaaatcaccaagatcatcagtctgggaaagaaaaagccgtccacagacgagcagacctcctcagctgaggaagatgttcccacctgcggctatctgaacgtgctctccaacagccgctggcgagagcgctggtgccgagtgaaagataacaagctcattttccacaaggacaggaccgacctgaagacccatattgtgtctattccgctccgtggctgcgaggtgatcccgggtttggattctaaacatcctctgacgttccggctgctgcgcaacggccaggaggttgcagtattggaggcatcttcttctgaagacatgggcaggtggattgggattttactcgcagagacgggatcgtccacagacccggaggctctgcactatgactacattgatgtggagatgtctgcaagtgtcattcagacagccaaacagaccttctgtttcatgaacaggcgtgttatatctgctaacccatatctagggggcacctccaacggctatgcccaccccagcgggacggcacttcattatgacgatgtcccgtgcatcaacggctcgctcaagggtaaaaagccccccgtggcgtctaatggggtcacaggaaaagggaagactctgagcagtcagccaaagaaagcggatcccgcggctgttgtgaaaaggacgggttcgaatgctgcccagtacaagtatggcaagaaccgggtagaagcagatgccaagcggctacagaccaaagaggaggagctgctgaagaggaaagaggccctgcggaataggctggcccagctccgcaaggaaagaaaagaccttcgagcggctattgaagtgaacgccggcaggaagccgcaggcgatcctggaggagaagctgaagcagctggaggaggagtgccggcagaaggaggcggagcgtgtcagcctggagctggagctgacggaggtcaaggagagcctgaagaaagcgctggcgggcggagtcaccctggggctggccatcgagcccaagtcagggacatcgagtccacagtctccagtgttccggcaccggaccctggaaaactcgcccatctccagctgtgacaccagtgacaccgagggccccgtgccggtgaacagcgcggccgtcttgaagaagagccaggctgccccgggcagctccccctgccgagggcatgtgctgcggaaggccaaggaatgggaattgaagaacgggacctaggggacagcagcaccactccagcctcagagactgcacacccccttgcctgtatcctcatctgtgtgacggcaggaagctctgcccagagtggcctcagctgcacgactccagaggctccacgactgagctctgaggccagtgcctgtcccccaggcccacttgtattctttctactgtaaaatggcgcctttaaaaaagatgtaagactttggttcccaactttgttaaaaaacaaaaaaagtgatggaaaggtgtttgaaaggatgctatcaggcgaagtgacatctttctaaccagataggccattgctttcaccgattccaacccagccctggactgaggatggatggttccatgagcccccagatgaaaacctggattcgacagggaagagcttggatccccggctgccccgggcatgagtgtggctggccatgggcactgggctcgccttgcacgcccaccaggagggccaggggcatacgcagtcctgacgtggactttgtgtcttggcgtaccctctccacgtgcccgtccccctcgtcatcagaggtaaaggcatgagaaaatcgcgctgtgaaacgttttttaccttttattattattttttggacagatgcttttgtcaccaagacatgaaaagctgccattcttcttaacctgttttttaagaatgtctttttactgtgtgtcacacttagatcaaggagttttcagaggttccttattttattaattaattatttgtgctgcttttgacaggtagcagagggttttaatacttcactaacagtacaccctcctcccccttgggctagcatccccagcccttaatccacgtcggagccggcaggtgcttctcacaccaggagcctctgggggcaggggctggctcctcgccgctgtacaggacgtttactgctgcttggggccagggcagtcctttgaaagggaagcaagttccggaatttgttccctcatggaactataacaccttagagcaagaggcaggaattccagggcctcctgcccctccccttcctccagcttgtcaagttaaaccactaatgtgttggtgggcctggccgtgctgcctctcctgagtagggtgggtgcaagtagggcctctttcccagctttcttgcttgccttctgtgcaatatttcttggagaatttggatgcctaaggcataaaggaaaaaaacagagttcatagtttgagctggtatttaccccctgggtttaaaaatcagattaacttcttggtctcccggaggccgctgtcaggaattctccagtacgggaagatgcagtcacttatgacgaaataagtcacccctgaacgcctccgcaggcagattcatggagaccagtcccagtagcaaccgccccttccccaagcatagaatacagaggtgccatacttggtgtaagccttccttctgtccactgtccagaatgtcctttttcacacagaggaagagatggcgggaaaacactgactgactccttgggtttcccccagacatactttcagagcgctagctgtgtggtgcacgggactttacagttaggattcatgtttaaggtggtggcgcttgagcccgcaagggccacgttggattactgaagcggaaagcggtctcccatgtataagaaaaagttttacaacacgaattggaagctgcttgagtccacctgaacttgaattgattgcaaagccatctgccgggaacggttttgggttcctgagccatcagccagcctttctttcccttctgatgcacgttcttcccggcctagtccgtggaggcccatcacaccagcatccgtgacgccagcttcccactaggacatccaggcaggtcgggcggggacgccacacgcctcagggccgtccgctgcactgccatagaggagccttccctccagtggtcacttcccaccattgcaggcagacttcccaccattgcaggcagctgggcccacttcatatcacacaaaatgaattttctataaagggaaaaaagtttgctcctccatagatttttgctgccttcgggtcaaatagtttccctggttgacgctcttgggccactctgattggcagcctgctttgccaaagagaactgtgtgaggttttggtggttagtaagagaagggggagatggccagcctgaagtcacggtcctggcccagcacaggtgggcagcagcgagctggcaggtgaggtgtgagcagcacgcaaagtgtcacgtgggccttagccgtcacccaccagcttcctcaccaggcaaggaggccccggagccttagcagggacttgcccgacggcacagctgattggtgctcgactcccagcatgcatattttgttgtaaaaacccaggtttgtaattcacatccacaaccctaagccttttaaagcggaggcaaacgttaacgaaccccatcgtggtccatggagcctttcaaaatgggtgccctggaggggcctgacagtgggttccttccatggcggatactcccttgaaatttcacattccatcaaagtttaaagcatttacttcagaataagttatttatgtgtatatgttcaataaatatagtttttaatttatatctgtacatattagacacacgcgagccatcttgccagaattctgacagtgatgtcattttattcccctcccaaaacatgccccaggaacaagcaccccttgcctctgtggcagctgcgagtgtgtctgatttcataacgaagccaggctgagggcaagaggcccgacaggcaggccagggtccccggagaagccggatttcttcgcaatgtcactggtacttagca

SEQ ID NO: 2 - protein, 730 aa, Homo sapiens [sequence omitted]

SEQ ID NO: 3 - DNA, 5040 bp, Homo sapiens
atggaagagttaatagttgaacttcgtctctttcttgaactcctggaccatgaatatctaacctcaactgtcagggagaaaaaggcagtgataaccaacattctgctaagaatacagtcatccaaaggttttgacgtgaaggaccatgctcagaagcaggagaccgctaacagcctgccagcccctcctcagatgcccctgccggagatccctcagccctggctgcctcctgacagtgggcctccaccattgccaacatcctccctcccagaaggttattatgaggaagctgtgccactgagccccggaaaagctccggaatacatcacatcaaattatgattccgatgcgatgagcagctcttatgagtcgtatgatgaagaggaggaggatgggaaggggaagaaaacccggcaccagtggccctccgaggaggcctccatggacctggtcaaggacgccaaaatctgcgccttcctgctgcggaagaagcggttcggccagtggaccaagttgctctgcgtcatcaaagacaccaaactgctgtgctataaaagttccaaggaccagcagcctcagatggaactgctactccaaggctgtaacattacgtacatcccgaaagacagcaaaaagaagaagcacgagctgaagattactcagcaggacacggacccgcttgttctcgccgtccagagcaaggaacaggccgggcagtggctgaaggtgatcaaagaagcctacagtggttgtagtggccccgtggattcagagtgtcctcctccaccaagctccccggtgcacaaggcagaactggagaagaaactgtcttcagagagacccagctcagatggggagggtgttgtggaaaatggaattaccacatgtaatggaaaggagcaagtgaagaggaagaaaagttccaaatcagaggccaagggcactgtgtcgaaagtcactgggaaaaaaatcaccaagatcatcagtctgggaaagaaaaagccgtccacagacgagcagacctcctcagctgaggaagatgttcccacctgcggctatctgaacgtgctctccaacagccgctggcgagagcgctggtgccgagtgaaagataacaagctcattttccacaaggacaggaccgacctgaagacccatattgtgtctattccgctccgtggctgcgaggtgatcccgggtttggattgtaaacatcctctgacgttccggctgctgcgcaacggccaggaggttgcagtattggaggcatcttcttctgaagacatgggcaggtggattgggattttactcgcagagacgggatcgtccacagacccggaggctctgcactatgactacattgatgtggagatgtctgcaagtgtcattcagacagccaaacagaccttctgtttcatgaacaggcgtgttatatctgctaacccatatctagggggcacctccaacggctatgcccaccccagcgggacggcacttcattatgacgatgtcccgtgcatcaacggctcgctcaagggtaaaaagccccccgtggcgtctaatggggtcacaggaaaagggaagactctgagcagtcagccaaagaaagcggatcccgcggctgttgtgaaaaggacgggttcgaatgctgcccagtacaagtatggcaagaaccgggtagaagcagatgccaagcggctacagaccaaagaggaggagctgctgaagaggaaagaggccctgcggaataggctggcccagctccgcaaggaaagaaaagaccttcgagcggctattgaagtgaacgccggcaggaagccgcaggcgatcctggaggagaagctgaagcagctggaggaggagtgccggcagaaggaggcggagcgtgtcagcctggagctggagctgacggaggtcaaggagagcctgaagaaagcgctggcgggcggagtcaccctggggctggccatcgagcccaagtcagggacatcgagtccacagtctccagtgttccggcaccggaccctggaaaactcgcccatctccagctgtgacaccagtgacaccgagggccccgtgccggtgaacagcgcggccgtcttgaagaagagccaggctgccccgggcagctccccctgccgagggcatgtgctgcggaaggccaaggaatgggaattgaagaacgggacctaggggacagcagcaccactccagcctcagagactgcacacccccttgcctgtatcctcatctgtgtgacggcaggaagctctgcccagagtggcctcagctgcacgactccagaggctccacgactgagctctgaggccagtgcctgtcccccaggcccacttgtattctttctactgtaaaatggcgcctttaaaaaagatgtaagactttggttcccaactttgttaaaaaacaaaaaaagtgatggaaaggtgtttgaaaggatgctatcaggcgaagtgacatctttctaaccagataggccattgctttcaccgattccaacccagccctggactgaggatggatggttccatgagcccccagatgaaaacctggattcgacagggaagagcttggatccccggctgccccgggcatgagtgtggctggccatgggcactgggctcgccttgcacgcccaccaggagggccaggggcatacgcagtcctgacgtggactttgtgtcttggcgtaccctctccacgtgcccgtccccctcgtcatcagaggtaaaggcatgagaaaatcgcgctgtgaaacgttttttaccttttattattattttttggacagatgcttttgtcaccaagacatgaaaagctgccattcttcttaacctgttttttaagaatgtctttttactgtgtgtcacacttagatcaaggagttttcagaggttccttattttattaattaattatttgtgctgcttttgacaggtagcagagggttttaatacttcactaacagtacaccctcctcccccttgggctagcatccccagcccttaatccacgtcggagccggcaggtgcttctcacaccaggagcctctgggggcaggggctggctcctcgccgctgtacaggacgtttactgctgcttggggccagggcagtcctttgaaagggaagcaagttccggaatttgttccctcatggaactataacaccttagagcaagaggcaggaattccagggcctcctgcccctccccttcctccagcttgtcaagttaaaccactaatgtgttggtgggcctggccgtgctgcctctcctgagtagggtgggtgcaagtagggcctctttcccagctttcttgcttgccttctgtgcaatatttcttggagaatttggatgcctaaggcataaaggaaaaaaacagagttcatagtttgagctggtatttaccccctgggtttaaaaatcagattaacttcttggtctcccggaggccgctgtcaggaattctccagtacgggaagatgcagtcacttatgacgaaataagtcacccctgaacgcctccgcaggcagattcatggagaccagtcccagtagcaaccgccccttccccaagcatagaatacagaggtgccatacttggtgtaagccttccttctgtccactgtccagaatgtcctttttcacacagaggaagagatggcgggaaaacactgactgactccttgggtttcccccagacatactttcagagcgctagctgtgtggtgcacgggactttacagttaggattcatgtttaaggtggtggcgcttgagcccgcaagggccacgttggattactgaagcggaaagcggtctcccatgtataagaaaaagttttacaacacgaattggaagctgcttgagtccacctgaacttgaattgattgcaaagccatctgccgggaacggttttgggttcctgagccatcagccagcctttctttcccttctgatgcacgttcttcccggcctagtccgtggaggcccatcacaccagcatccgtgacgccagcttcccactaggacatccaggcaggtcgggcggggacgccacacgcctcagggccgtccgctgcactgccatagaggagccttccctccagtggtcacttcccaccattgcaggcagacttcccaccattgcaggcagctgggcccacttcatatcacacaaaatgaattttctataaagggaaaaaagtttgctcctccatagatttttgctgccttcgggtcaaatagtttccctggttgacgctcttgggccactctgattggcagcctgctttgccaaagagaactgtgtgaggttttggtggttagtaagagaagggggagatggccagcctgaagtcacggtcctggcccagcacaggtgggcagcagcgagctggcaggtgaggtgtgagcagcacgcaaagtgtcacgtgggccttagccgtcacccaccagcttcctcaccaggcaaggaggccccggagccttagcagggacttgcccgacggcacagctgattggtgctcgactcccagcatgcatattttgttgtaaaaacccaggtttgtaattcacatccacaaccctaagccttttaaagcggaggcaaacgttaacgaaccccatcgtggtccatggagcctttcaaaatgggtgccctggaggggcctgacagtgggttccttccatggcggatactcccttgaaatttcacattccatcaaagtttaaagcatttacttcagaataagttatttatgtgtatatgttcaataaatatagtttttaatttatatctgtacatattagacacacgcgagccatcttgccagaattctgacagtgatgtcattttattcccctcccaaaacatgccccaggaacaagcaccccttgcctctgtggcagctgcgagtgtgtctgatttcataacgaagccaggctgagggcaagaggcccgacaggcaggccagggtccccggagaagccggatttcttcgcaatgtcactggtacttagca

SEQ ID NO: 4 - protein, 730 aa, Homo sapiens
MEELIVELRLFLELLDHEYLTSTVREKKAVITNILLRIQSSKGFDVKDHAQKQETANSLPAPPQMPLPEIPQPWLPPDSGPPPLPTSSLPEGYYEEAVPLSPGKAPEYITSNYDSDAMSSSYESYDEEEEDGKGKKTRHQWPSEEASMDLVKDAKICAFLLRKKRFGQWTKLLCVIKDTKLLCYKSSKDQQPQMELLLQGCNITYIPKDSKKKKHELKITQQDTDPLVLAVQSKEQAGQWLKVIKEAYSGCSGPVDSECPPPPSSPVHKAELEKKLSSERPSSDGEGVVENGITTCNGKEQVKRKKSSKSEAKGTVSKVTGKKITKIISLGKKKPSTDEQTSSAEEDVPTCGYLNVLSNSRWRERWCRVKDNKLIFHKDRTDLKTHIVSIPLRGCEVIPGLDCKHPLTFRLLRNGQEVAVLEASSSEDMGRWIGILLAETGSSTDPEALHYDYIDVEMSASVIQTAKQTFCFMNRRVISANPYLGGTSNGYAHPSGTALHYDDVPCINGSLKGKKPPVASNGVTGKGKTLSSQPKKADPAAVVKRTGSNAAQYKYGKNRVEADAKRLQTKEEELLKRKEALRNRLAQLRKERKDLRAAIEVNAGRKPQAILEEKLKQLEEECRQKEAERVSLELELTEVKESLKKALAGGVTLGLAIEPKSGTSSPQSPVFRHRTLENSPISSCDTSDTEGPVPVNSAAVLKKSQAAPGSSPCRGHVLRKAKEWELKNGT

SEQ ID NO: 5 - DNA, 26 bp, Artificial Sequence
  PCR Primer
gaaagaaaaagccgtccacagacgag

SEQ ID NO: 6 - DNA, 23 bp, Artificial Sequence
  PCR Primer
ttgagcgagccgttgatgcacgg